(19) United States
(12) Patent Application Publication
(10) Pub. No.: US 2023/0323352 A1
(43) Pub. Date: Oct. 12, 2023

(54) EXOSOME SECRETED FROM GENE-MODIFIED CELLS WITH LONG NON-CODING RIBONUCLEIC ACIDS AND APPLICATION THEREOF

(71) Applicant: WENZHOU MEDICAL UNIVERSITY, Wenzhou (CN)

(72) Inventors: Wei LI, Wenzhou (CN); Lin LIU, Wenzhou (CN); Xudong HU, Wenzhou (CN)

(21) Appl. No.: 18/332,721

(22) Filed: Jun. 10, 2023

Related U.S. Application Data
(63) Continuation of application No. PCT/CN2022/106965, filed on Jul. 21, 2022.

(30) Foreign Application Priority Data
Dec. 3, 2021 (CN) 202111467233.9

(51) Int. Cl. C12N 5/071; C12N 15/113; C12N 15/86; A61K 35/22

(52) U.S. Cl. CPC C12N 15/113; A61K 35/22; C12N 5/0686; C12N 15/86; C12N 2510/02; C12N 2740/15043

(57) ABSTRACT

Disclosed is an exosome secreted from gene-modified cells with long non-coding ribonucleic acids (lncRNA) elevated in non-alcoholic fatty liver (lncENAF) and application thereof, belonging to the technical field of cell biology. The exosome is secreted by a cell strain of human embryonic kidney 293T cells (HEK-293T) obtained by genetic engineering, and the cell strain of HEK-293T stably expresses lncENAF, where the lncENAF has a nucleotide sequence as shown in SEQ ID NO: 1.

### CROSS-REFERENCE TO RELATED APPLICATIONS

This application is a continuation of PCT/CN2022/106965, filed Jul. 21, 2022, and claims priority of Chinese Patent Application No. 202111467233.9, filed on Dec. 3, 2021, the entire contents of which are incorporated herein by reference.

### INCORPORATION BY REFERENCE STATEMENT

This statement, made under Rules 77(b)(5)(ii) and any other applicable rule, incorporates into the present specification of an XML file for a “Sequence Listing XML” (see Rule 831(a) ), submitted via the USPTO patent electronic filing system or on one or more read-only optical discs (see Rule 1.52(e)(8) ), identifying the names of each file, the date of creation of each file, and the size of each file in bytes as follows:
File name: 20230509_sequence_347_007_2023_2795
Creation date: May 9, 2023
Byte size: 22,172

### TECHNICAL FIELD

The present application relates to the technical field of cell biology, and in particular to an exosome secreted from gene-modified cells with long non-coding ribonucleic acids (lncRNA) and an application thereof.

### BACKGROUND

Long non-coding ribonucleic acid (lncRNA) is a class of non-coding sequences with a transcriptional length of more than 200 nucleotides (nt) that encode few or no proteins for lack of a valid open reading frame. It was previously considered to have no biological function and existed only as a by-product of the transcriptional process owing to the shallow research on lncRNA. As sequencing technology for molecular biology continues to develop, lncRNA has been found to be capable of gene regulation at different levels, such as epigenetic regulation, transcriptional regulation and post-transcriptional regulation, and the regulatory functions of lncRNA are therefore gaining growing attention and research.

Exosomes are extracellular vesicles with a particle size of 60 - 200 nanometers (nm); they are secreted by almost all cells and capable of containing a number of complex substances (for instance, nucleic acids, proteins, lipids, etc.), making it possible for exosomes to participate in intercellular signaling as an important mediator of intercellular communication. Studies have suggested that LncRNA HOX transcript antisense RNA (HOTAIR) promotes exosome secretion by mediating the expression of exosome-formation-associated proteins, enriching the understanding of the regulation of exosome secretion by LncRNA to some extent. Nevertheless, relevant reports on the effects of exosomes regulated by LncRNA on macrophage cytokines are still scarce.

### SUMMARY

The present application provides an exosome secreted from gene-modified cells with long non-coding ribonucleic acids (lncRNA) and an application thereof, so as to solve the problems existing in the prior art. The exosome is a potential inhibitor of cytokines by effectively inhibiting lipopolysaccharide (LPS)-induced macrophage cytokine production, providing a new direction for cytokine storm and treatment of autoimmune diseases.

In order to achieve the above objectives, the present application provides the following technical schemes:

the present application provides an exosome inhibiting macrophage cytokines; the exosome is secreted by a cell strain of human embryonic kidney 293T cells (HEK-293T); the cell strain of HEK-293T stably expresses a lncRNA elevated in non-alcoholic fatty liver (lncENAF), and the lncENAF has a nucleotide sequence as shown in SEQ ID NO:1.

The present application also provides an application of the exosome in preparing a medication for inhibiting increasing cytokines levels induced by LPS.

The present application also provides an application of the exosome in preparing a medication for inhibiting cytokine storms or treating autoimmune diseases.

Optionally, the autoimmune diseases include sepsis, viral pneumonia, rheumatoid arthritis, encephalitis, pulmonary fibrosis, steatohepatitis and multiple sclerosis.

Optionally, the exosome achieves inhibiting cytokine storms or treating autoimmune diseases by inhibiting LPS-induced increasing of cytokines levels.

Optionally, the cytokines include interleukin-6 (IL-6) and interleukin-1 beta (IL-1β).

The present application also provides a medication for inhibiting increasing cytokines levels induced by LPS, and the medication includes the exosome and a pharmaceutically or immunologically combinable carrier or auxiliary material.

The present application also provides a medication for inhibiting cytokines or treating autoimmune diseases, and the medication includes the exosome and a pharmaceutically or immunologically combinable carrier or auxiliary material.

The present application also provides a method for constructing the cell strain of HEK-293T, including:

S1, obtaining a gene sequence of lncENAF and constructing a lentiviral vector for stably expressing the lncENAF;

S2, mixing HEK-293T cells with the lentiviral vector for lentiviral plasmid transfection to obtain virus solution; and

S3, mixing the virus solution with the HEK-293T cells for culture, and obtaining the cell strain of HEK-293T stably expressing lncENAF through antibiotic screening.

The present application also provides a usage of the exosome inhibiting macrophage cytokines, including steps as follows:

constructing a cell strain of HEK-293T stably expressing lncENAF by the method for constructing the cell strain of HEK-293T, then culturing to collect a culture solution, followed by centrifugation to collect exosomes secreted by the cell strain of HEK-293T; co-incubating the exosomes with macrophages, and detecting expression levels of cytokines of the macrophages.

The present application discloses the following technical effects:

a noncoding RNA elevated in non-alcoholic fatty liver named lncENAF is found by constructing a mouse model of nonalcoholic steatohepatitis according to the present application, then a cell strain stably expressing lncENAF is constructed by genetic engineering; the exosome secreted by this cell strain is incubated with macrophages, and it is found that the exosome significantly inhibits the production of cytokine IL-6 induced by LPS, suggesting that the exosome provided by the present application is a potential cytokine inhibitor, therefore providing data to support the suppression of cytokines and offering new strategies to combat cytokine storms and related autoimmune diseases caused by LPS-induced elevation of cytokines.

### BRIEF DESCRIPTION OF THE DRAWINGS

In order to explain the embodiments of the present application or the technical scheme in the prior art more clearly, the drawings needed in the embodiments are briefly introduced below. Obviously, the drawings described below are only some embodiments of the present application, and other drawings may be obtained according to these drawings without creative work for ordinary people in the field.

FIG. 1 shows results of electrophoresis verification after polymerase chain reaction (PCR) of a pCDH-GFP-lncENAF plasmid bacterial solution.

FIG. 2 shows sequencing comparison results between a full-length lncRNA elevated in non-alcoholic fatty liver (lncENAF) and recombinant plasmid, where a nucleotide sequence of Query is shown in SEQ ID No: 13 and a nucleotide sequence of Sbjct is shown in SEQ ID No: 1.

FIG. 3 is a map of pCDH-GFP-lncENAF vector.

FIG. 4A is a fluorescence diagram of cell strain of human embryonic kidney 293T cells-long non-coding RNA elevated in nonalcoholic fatty liver (HEK-293T-lncENAF).

FIG. 4B shows Cq value of lncENAF in HEK-293T-lncENAF cell strain detected by quantitative-PCR (qPCR).

FIG. 5A illustrates a process of extracting exosome.

FIG. 5B shows results of electron microscopic identification of the exosome.

FIG. 5C illustrates results of particle size and concentration analysis of the exosome.

FIG. 5D illustrates qualitative analysis of exosome feature proteins (TSG101 and CD9).

FIG. 6A shows the exosome entering cells.

FIG. 6B shows the exosome inhibiting lipopolysaccharide (LPS)-induced interleukin-6 (IL-6) messenger ribonucleic acid (mRNA) synthesis.

FIG. 6C shows extracellular IL-6 release.

FIG. 6D shows the exosome inhibiting LPS-induced interleukin-1 beta (IL-1β) mRNA synthesis.

FIG. 6E shows extracellular IL-1β release.

FIG. 7 shows a process of a method for constructing the cell strain of HEK-293T.

### DETAILED DESCRIPTION OF THE EMBODIMENTS

A number of exemplary embodiments of the present application are now be described in detail, and this detailed description should not be considered as a limitation of the present application, but should be understood as a more detailed description of certain aspects, characteristics and embodiments of the present application.

It should be understood that the terminology described in the present application is only for describing specific embodiments and is not used to limit the present application. In addition, for the numerical range in the present application, it should be understood that each intermediate value between the upper limit and the lower limit of the range is also specifically disclosed. The intermediate value within any stated value or stated range and every smaller range between any other stated value or intermediate value within the stated range are also included in the present application. The upper and lower limits of these smaller ranges may be independently included or excluded from the range.

Unless otherwise specified, all technical and scientific terms used herein have the same meaning as commonly understood by one of ordinary skill in the art to which this present application relates. Although the present application only describes the preferred methods and materials, any methods and materials similar or equivalent to those described herein can also be used in the practice or testing of the present application. All documents mentioned in this specification are incorporated by reference to disclose and describe methods and/or materials related to the documents. In case of conflict with any incorporated document, the contents of this specification shall prevail.

It is obvious to those skilled in the art that many improvements and changes can be made to the specific embodiments of the present application without departing from the scope or spirit of the present application. Other embodiments will be apparent to the skilled person from the description of the present application. The specification and example of this application are only exemplary.

The terms “including”, “comprising”, “having” and “containing” used in this article are all open terms, which means including but not limited to.

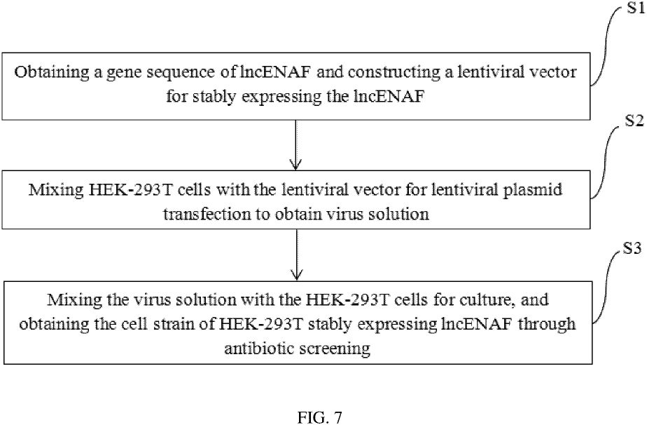

In a previous research (Chen Q, Xiong C, Jia K, et al. Hepatic transcriptome analysis from HFD-fed mice defines a long noncoding RNA regulating cellular cholesterol levels. J Lipid Res. 2019;60(2):341-352), a significantly up-regulated non-coding RNA, (NONCODE: NONMMUG027912.3) is discovered in a constructed mouse model of nonalcoholic steatohepatitis after ribonucleic acid (RNA) sequencing, which is named as long non-coding RNA elevated in nonalcoholic fatty liver (lncENAF), and a cell strain of human embryonic kidney 293T cells (HEK-293T) stably expressing the lncENAF is constructed using transgenic technology following a method for constructing the cell strain of HEK-293T as shown in FIG. 7, including:
S1, obtaining a gene sequence of lncENAF and constructing a lentiviral vector for stably expressing the lncENAF;
S2, mixing HEK-293T cells with the lentiviral vector for lentiviral plasmid transfection to obtain virus solution; and
S3, mixing the virus solution with the HEK-293T cells for culture, and obtaining the cell strain of HEK-293T stably expressing lncENAF through antibiotic screening.

The HEK-293T-lncENAF cell strain is then cultured, and the cell culture medium modified by lncENAF gene is collected and subjected to ultra-centrifugation to collect secreted exosomes; then the exosomes are co-incubated with macrophages to discover that the exosome can significantly inhibit macrophage cytokines induced by lipopolysaccharide (LPS), including interleukin-6 (IL-6).

### Embodiment 1

### 1. Construction of Lentiviral Vector Overexpressing lncENAF

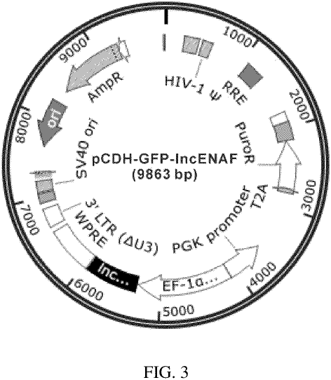

The pMD-T18-lncENAF plasmid and strain are available in the study group. The pCDH-GFP-lncENAF plasmid is constructed by linking lncENAF (the nucleotide sequence of lncENAF is shown in SEQ ID NO:1) to the pCDH-GFP plasmid through the design of homology arm primers (Xba I and Sal I) (see FIG. 3 for plasmid map and SEQ ID NO:2 for nucleotide sequence of pCDH-GFP-lncENAF). The homology arm primers are shown in the accompanying diagram (see Table 1) and are constructed as follows:

TABLE 1

Homology arm primer sequence

| | | |
|---|---|---|
| Name | Forward (5′→3′) | Reverse (5′→3′) |
| Sequence of homology arm primer | tttcaggtgtcgtgatctagaATTGTACAC CATGCAGACAAAGCG (SEQ ID NO:3) | atccagaggttgattgtcgacGGCCTTGAGGT CATACTCAAGC (SEQ ID NO:4) |

(1) amplification of lncENAF by polymerase chain reaction (PCR): lncENAF is amplified by PCR with amplification template of pMD-T18-lncENAF, the amplification system is as follows:

TABLE 2

Amplification system

| Reagent | Volume (µL) |
|---|---|
| template | 1 |
| Forward | 0.5 |
| Reverse | 0.5 |
| 2×Taq enzyme | 5 |
| ddH₂O | 3 |

(2) agarose powder of 0.5 gram (g) is weighed and added into a clean conical flask, then 50 milliliters (mL) of Tris-acetate-ethylene diamine tetraacetic acid (TAE) buffer solution is added, followed by heating for melting in a microwave oven, then the conical flask is washed with running water and cooled to about 40° C. (°C), and 0.5 microliter (µL) of Gold View I nucleic acid dye is added, followed by mixing well and pouring into a gel tank, then a comb is inserted to stand for 30 minutes (min), the comb is removed after the agarose gel is solidified, and the gel is transferred into an electrophoresis tank containing an appropriate amount of 1×TAE;
(3) after PCR, 1 µL of 10×loading buffer is added to each PCR reaction system, followed by mixing well by a sample gun and transferring to sample adding wells in the gel, and then electrophoresis is performed at 120 Volts (V) for 25 min;
(4) the gel is placed in a gel imaging system after electrophoresis, the target band is cut off under ultraviolet irradiation, and then 0.2 g of it is put into a 1.5 mL sterile EP tube;
(5) 200 µL of binding buffer is added, and the gel is melted in water bath at 50° C., with shaking once every 2 min;
(6) the liquid obtained after above steps is transferred into a collection column of HiBind® DNA Mini Column, followed by centrifugation at room temperature for 1 min at 20,000 g, with waste liquid discarded;
(7) binding buffer of 300 µL is added, followed by centrifugation at 20,000 g at room temperature for 1 min, with waste liquid discarded;
(8) washing buffer of 700 µL is added, followed by centrifugation at 20,000 g at room temperature for 1 min, with waste liquid discarded, and the present step is repeated once;
(9) after centrifugation at 20,000 g for 1 min at a room temperature, the collection column of HiBind® DNA Mini Column is transferred to a clean 1.5 mL EP tube and left uncapped for 2 min at a room temperature;
(10) ddH₂O of 30 µL is added and stood at room temperature for 2 min, then centrifuged at a room temperature for 1 min at 20,000 g; at this time, what is collected in the EP tube is the lncENAF; then the lncENAF is detected by a DeNovix DS-11+Spectrophotometer in terms of concentration and purity of the lncENAF, and the subsequent operation is carried out after recording;
(11) pCDH-GFP plasmid is recovered by double digestion, and the double digestion system is as follows:

TABLE 3

Double enzyme digestion system

| | |
|---|---|
| Reagent | Dosage |
| pCDH-GFP plasmid | 1 microgram (µg) |
| Xba I | 1 µL |
| Sal I | 1 µL |
| 10×NEB Buffer | 2 µL |
| ddH₂O | up to 20 µL |

after mixing, the mixture is reacted at 37° C. for 2 h;
(12) after the reaction, electrophoresis and gel cutting are carried out, and the steps are the same as above;
(13) the following seamless cloning system (using Heyuan seamless cloning kit) is prepared:

TABLE 4

Seamless Cloning System

| | |
|---|---|
| Reagent | Dosage |
| 5× seamless cloning buffer | 2 µL |
| PCDH-GFP double enzyme digestion product | 0.015 pmol |
| LncENAF of full length | 0.030 pmol |
| Seamless cloning enzyme | 1 µL |
| ddH₂O | up to 10 µL |

a mixture is obtained by mixing well and then subjected to reaction at 37° C. for 30 min;
(14) DH-5α competent cells are taken out under -80° C. and thawed on ice, then 50 µL of it is added into a clean 1.5 mL EP tube, followed by adding 10 µL of seamless cloning system and standing on ice for 30 min, and immediately subjected to water-bathing at 42° C. for 45 seconds (s); then it is placed back on ice and stood for 3 min, followed by adding 940 µL of LB medium and shaking culture at 37° C. for 2 h;
(15) after the end of culture, it is centrifuged at 3,000 g under a room temperature for 5 min, and 900 µL of the supernatant is discarded, and the remaining 100 µL is mixed well by a sample gun and added into the center of a plate containing Ampicillin resistance, and then the bacterial liquid is spread evenly with a sterile coating rod and cultured overnight at 37° C.;
(16) after overnight culture, a single colony is picked up by an inoculation ring and shaking-cultured in 400 µL of LB bacterial culture medium containing Ampicillin resistance at 37° C. for 2 h;
(17) after the shaking-culturing of 2 h, 1 µL of the bacterial liquid is taken as a template to carry out bacterial liquid PCR so as to identify whether the pCDH-GFP-lncENAF plasmid is successfully constructed, where the system is as follows:

TABLE 5

PCR identification system

| Reagent | Volume (µL) |
|---|---|
| Bacterial liquid | 1 |
| Forward | 0.5 |
| Reverse | 0.5 |
| 2×Taq enzyme | 5 |
| ddH₂O | 3 |

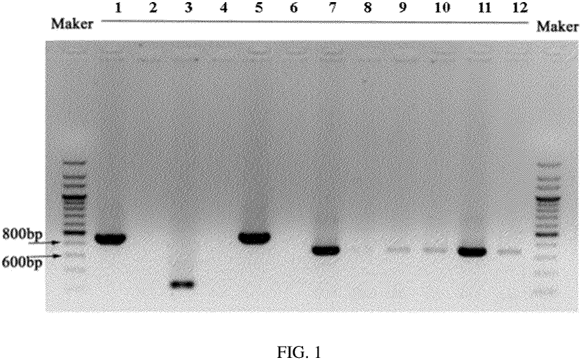

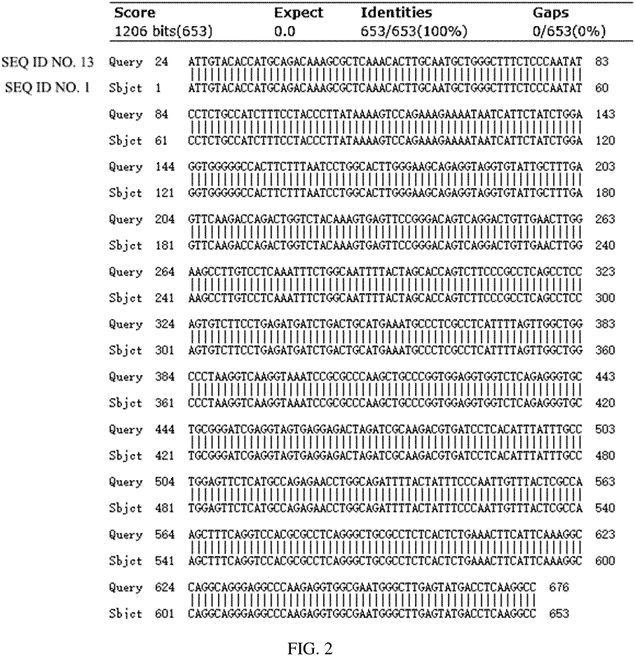

after mixing well, the bacterial liquid is put into a PCR instrument for reaction;
(18) after the PCR reaction is finished, agarose gel electrophoresis is carried out, with the same steps as above, and exposure identification is carried out after the electrophoresis, with results showing that the PCR product fragments of the seventh tube and the eleventh tube are of 600 base pairs (bp) -800 bp, and the bands are clear and bright (see FIG. 1); then the seventh tube of bacterial liquid is selected and sent to Shanghai Sunny Biotechnology Co., Ltd. for Sanger sequencing, with results of sequencing comparison showing that the full length of lncENAF is 100% matched with the recombinant plasmid (see FIG. 2, where a nucleotide sequence of Query is shown in SEQ ID No: 13 and a nucleotide sequence of Sbjct is shown in SEQ ID No: 1), suggesting that the pCDH-GFP-lncENAF plasmid is successfully constructed (see FIG. 3 for the plasmid map).

### 2. Extraction of Lentivirus Expression Plasmids and Packaging Plasmids

The lentivirus packaging plasmids are psPAX and pMD2.G, respectively; the extraction follows the procedure described in the OMEGA Endotoxin Removal Plasmid Extraction Kit (D6948-01), the details of which are as follows:
(1) the ultraviolet lamp of the ultra-clean ultraviolet table is turned on for irradiation of 30 min;
(2) a 50 mL EP tube is taken and added with 30 mL LB medium, 30 µL Ampicillin (Amp+) (diluted at 1: 1,000), and finally 50 µL bacterial liquid, followed by culture at 37° C. and 250 rpm for 16 h;
(3) the bacterial liquid is taken out and centrifuged for 1 min at 10,000 g, with supernatant discarded;
(4) a Solution I of 600 µL is added and transferred to a 2 mL EP tube after blowing and suspending the bacterial precipitation, followed by violently shaking by a vortex instrument for 1 min;
(5) then a Solution II of 600 µL is added, and the 2 mL EP is gently inverted for 6 times, and stood at room temperature for 2 min;
(6) 300 µL of pre-cooled N3 Buffer is added, blended by inverting until a white turbid precipitate is formed and left to stand for 2 min at room temperature;
(7) then the tube is centrifuged at 20,000 g under room temperature for 5 min, the supernatant is sucked to a new 2 mL EP tube and added with ⅒ volume of ETR Solution, followed by inverting for mixing well and standing on ice for 10 min (upside down for 3 times every 2 min), standing for 2 min at 42° C., and centrifuging at 20,000 g under room temperature for 5 min;
(8) the supernatant is taken to another new 2 mL EP tube, and added with ½ volume of anhydrous ethanol, followed by inverting for mixing well and standing under room temperature for 2 min;
(9) a Hiband® DNA Mini Column is taken and added with 700 µL of liquid obtained after step (8), followed by centrifuging at 20,000 g under room temperature for 1 min;
(10) the liquid in the collection column is then discarded and the operations of (9) is repeated until the liquid of step (8) is completely centrifuged;
(11) the liquid in the collection column is again discarded and 500 µL HBC Buffer is added, followed by centrifuging at 20,000 g under room temperature for 1 min;
(12) the collection column is discarded, and the Hiband® DNA Mini Column is transferred to a new 1.5 mL EP tube, followed by standing at room temperature for 3 min;
(13) diethyl pyrocarbonate (DEPC) water of 50 µL is added and stood under room temperature for 3 min, then centrifuged at 20,000 g under room temperature for 1 min;
(14) the Hiband® DNA Mini Column is discarded, the plasmids are collected and detected by a Nano Drop microvolume spectrophotometer in terms of concentration and purity, and preserved at -20° C. after marking;

### 3. Lentivirus Package

(1) the cultured HEK-293T cells are taken out and 100×10⁴ cells are inoculated into a culture dish with a diameter of 6 centimeters (cm), two culture dishes in total; and the culture dishes are subjected to shaking culture by a cross method to make the cells grow uniformly;
(2) lentivirus plasmid transfection is performed when the cell growth density reaches 70%;
(3) two 1.5 mL EP tubes are taken and labeled as a tube A and a tube B, with 500 µL Opti-MEM culture medium added to each tube;
(4) the tube A is added with 10 µL of lipofectamine 3000 reagent, followed by blowing and mixing evenly, and standing at a room temperature for 1 min;
(5) the tube B is 10 µL of P3000, 4 µg of pCDH-lncENAF plasmid (pCDH-GFP plasmid is added to the control group), 3 µg of psPAX2 plasmid and 1 µg of pMD2.G plasmid, followed by blowing and mixing evenly, and standing at room temperature for 1 min;
(6) all the liquid in tube B is transferred to the tube A, followed by blowing and mixing evenly, and standing at room temperature for 15 min;
(7) the mixture obtained after step (6) is drip-added into the HEK-293T cells, and cultured in an incubator for 12 h, then replaced with DMEM medium, and the culture is continued for 24 h and 48 h, and the virus supernatant is collected; and
(8) the collected virus supernatant is filtered by a filter with pore size of 0.45 µm, and sub-packaged into 1.5 mL EP tubes, with 1 mL for each tube, and stored at -80° C.

### 4. Screening of Lethal Puromycin Concentration in HEK-293T Cells and Construction of HEK-293T-lncENAF Cell Strain

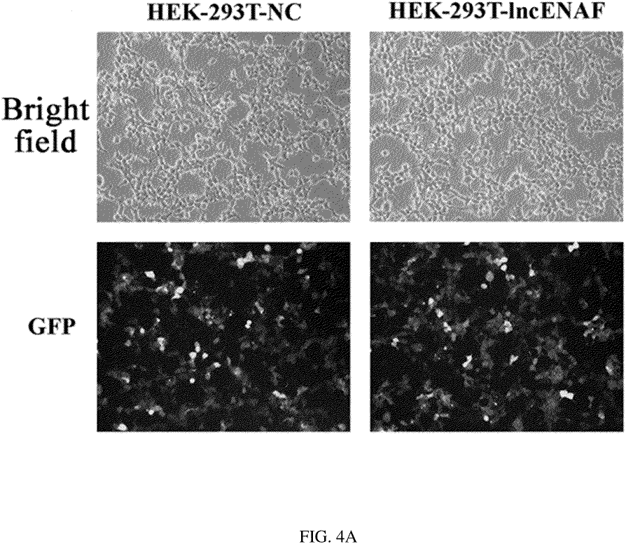

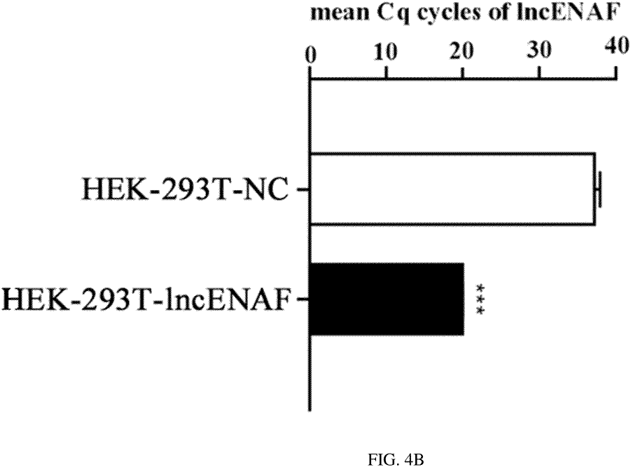

(1) HEK-293T cells are inoculated into 6-well plates, with 25×10⁴ cells per well;
(2) the medium is changed to Dulbecco’s Modified Eagle Medium (DMEM) containing puromycin when the cell density is about 50%, and puromycin with a gradient of four concentration of 0 µg/mL, 0.2 µg/mL, 0.3 µg/mL and 0.4 µg/mL is used respectively;
(3) after continuous culture of 72 h, it is found that the maximum lethal puromycin concentration of HEK-293T cells is 0.4 µg/mL;
(4) after determining the optimal puromycin concentration, HEK-293T cells are inoculated into 6-well plates, with 25×10⁴ cells in each well, and cultured for 12 h until the cells adhered to the wall;
(5) 300 µL virus solution is added to each well to infect cells for 12 h, then it is replaced by fresh DMEM medium to continue the culture for 72 h; and
(6) the genes carried by the virus are randomly integrated into HEK-293T cells after 72 hours of virus infection, at which time the medium is replaced with puromycin medium containing 0.4 µg/mL and culture is continued for 7 days, with fresh puromycin-containing DMEM medium replaced once a day; the cells that survived after 7 days are screened as a cell strain of HEK-293T that stably expresses lncENAF, named HEK-293T-lncENAF, and green fluorescence can be seen under an inverted fluorescence microscope (see FIG. 4A).

### 5. Fluorescence Quantitative-PCR (qPCR)

The system is loaded using SYBR Premix Ex TaqTM II kit with reference to the instructions (RR820A, Takara), in a BioRad instrument, according to the following procedures: 95° C. for 3 min, (95° C. for 5 s, 60° C. for 30 s, 72° C. for 40 s, 40 cycles), 72° C. for 5 min, 95° C. for 15 s, 60° C. for 1 min, 95° C. for 15 s. The difference of Cq values of lncENAF in the HEK-293T-lncENAF cell strain and the control cell strain are observed at the end of the reaction, with results showing that the Cq value of lncENAF in the HEK-293T-lncENAF cell strain is significantly lower than that of the control strain (see FIG. 4B). qPCR primer sequences involved in the experiments are synthesized by Tsingke Biotechnology Co., Ltd., and the sequences are shown in the accompanying figures (see Table 6).

TABLE 6

Primer sequences

| | | |
|---|---|---|
| Gene name | Forward (5′→3′) | Reverse (5′→3′) |
| β-actin | CATCCGTAAAGACCTCTATGCCAA C (SEQ ID NO:5) | ATGGAGCCACCGATCCACA (SEQ ID NO:6) |
| lncENA F | GGAAGCAGAGGTAGGTGTAT (SEQ ID NO:7) | GGCTTCCAAGTTCAACAGTC (SEQ ID NO:8) |
| IL-6 | CGGCCTTCCCTACTTCACAA (SEQ ID NO:9) | TTGCCATTGCACAACTCTTTT C (SEQ ID NO:10) |
| IL-1β | GAAATGCCACCTTTTGACAGTG (SEQ ID NO:11) | TGGATGCTCTCATCAGGACA G (SEQ ID NO:12) |

### 6. Isolation, Purification and Identification of Exosomes

### 6.1 Acquisition, Separation and Purification of Exosomes

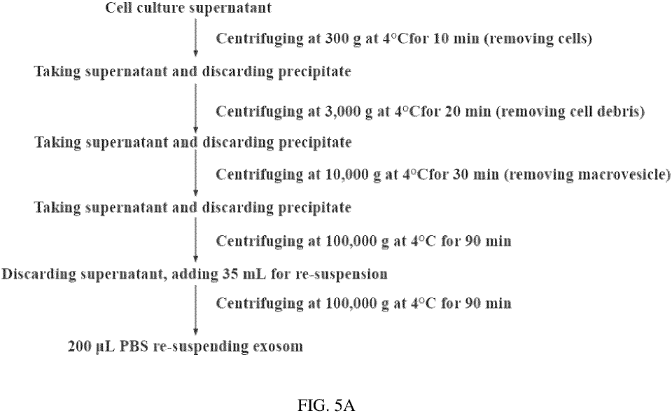

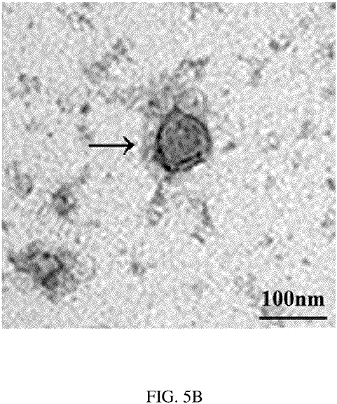

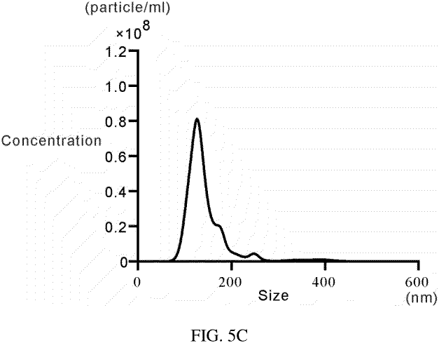

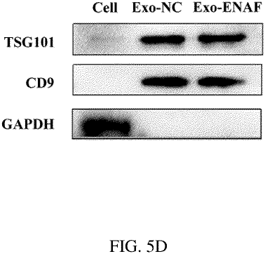

(1) HEK-293T-lncENAF cell strain is inoculated into a 15 cm culture dish, each culture dish is inoculated with 2.0×10⁶ cells, and then cultured in a carbon dioxide incubator at a constant temperature of 37° C.;
(2) the culture medium is replaced by a DMEM medium without serum when the confluent degree of cells reaches 80%, and continue to culture for 24 h;
(3) the cell culture supernatant is collected to a 50 mL EP tube, and the exosomes are collected according to the exosomes extraction process (see FIG. 5A); the EP tube is firstly centrifuged at 300 g at 4° C. for 10 min;
(4) the supernatant is transferred to a new 50 mL EP tube, with precipitate discarded, followed by centrifugation at 3,000 g and 4° C. for 20 min;
(5) the supernatant obtained after (4) is transferred to a new 50 mL EP tube, with precipitate discarded, then the EP tube is centrifuged at 4° C. and 10,000 g for 30 min;
(6) the supernatant obtained after (5) is transferred to a SW 32Ti ultracentrifuge tube, and centrifuged at 4° C. and 100,000 g for 90 min;
(7) the supernatant obtained after (6) is discarded, and the precipitate is re-suspended after adding with 35 mL PBS, and centrifuged at 4° C. and 100,000 g for 90 min; and
(8) the supernatant obtained after (7) is discarded, the exosomes are re-suspended after adding with 200 µL PBS, filtered with filter of 0.22 µm pore size and stored at -80° C.

### 6.2 Identification of Exosomes

### 6.2.1 Observation of Negatively Dyed Exosomes by Transmission Electron Microscope (TEM)

The exosomes extracted by PBS re-suspension are dripped on a copper mesh with a pore size of 2 nanometers (nm), and allowed to stand at room temperature for 2 min. The liquid is drained by the side of the filter screen of filter paper, and negatively stained with 2% phosphotungstic acid solution at room temperature for 2 min. The negative dyeing solution is drained by filter paper, dried at room temperature, and photographed by electron microscope. The vesicle with a size of about 100 nm as indicated by the arrow is the exosome (see FIG. 5B).

### 6.2.2 Nanoparticle Tracking Analysis (NTA) of Exosomes Against Particle Size and Concentration

The isolated exosome samples are diluted with PBS and 500 µL of the samples are taken and diluted 10-fold and injected into a nanoparticle tracking analyzer. A laser is passed through the samples and scattered light is collected through a microscope equipped with a camera to capture the Brownian motion of the exosomes, and then the Stokes-Einstein equation is used to estimate the particle size and number by measuring the average velocity of the particles; the results show that the average particle size of exosomes is approximately 144 nm, with 4.39 × 10⁸ exosomes vesicles per 1 mL (see FIG. 5C).

### 6.2.3 Marker Protein Detection of Exosomes by Western Blot

(1) gel preparation: a 12% separating gel is prepared and added between thick and thin plates after fully mixing, the upper layer is flattened with pressure and left at room temperature for about 30 min, the upper layer of water is discarded; a 5% concentrating gel is prepared and added between thick and thin plates after thorough mixing, where a comb is inserted to avoid air bubbles, and the electrophoresis can be carried out after the concentrate gel has solidified;
(2) electrophoresis: the prepared gels are placed in the electrophoresis bath, with 1× electrophoresis buffer added; then the comb is pulled out vertically and 20 µg of protein samples are sampled per well; the concentrating gel is electrophoresed at 70 Volts (V) for 40 min and the separating gel is electrophoresed at 110 V for 60 min;
(3) membrane transfer: polyvinylidene fluoride (PVDF) membranes of suitable size are obtained by cutting, then activated in methanol for 90 s and then transferred to the transfer buffer for equilibration; after the electrophoresis is completed, the gel is cut according to the position of the protein marker, the gel is placed in the order of black transfer clip at the bottom, sponge, filter paper, gel, PVDF membrane, filter paper and sponge, the transfer clip is fixed and placed in the transfer tank with the transfer buffer and ice pack added, and the transfer instrument is switched on and electrophoresis is carried out at a constant current of 300 mA for 60 min;
(4) sealing: at the end of the transfer, the PVDF membrane is carefully removed with forceps and placed in 5% skimmed milk and sealed with gentle shaking at room temperature for 2 h;
(5) incubation of primary antibody: after sealing, the sealing solution is discarded and the bands are washed with 1×TBST for 5 min, three times in total; the bands with molecular weight of the target protein are cut off according to the position of the protein marker, absorbed by filter paper, placed in the corresponding diluted antibody and incubated overnight at 4° C. in a shaker;
(6) incubation of secondary antibody: the bands incubated overnight are taken out and washed with 1×TBST for 5 min, for a total of three times, and the bands are placed in the appropriate secondary antibody according to the source of the primary antibody and incubated for 1 h with gentle shaking at room temperature on a shaker;
(7) development: the bands are removed from the secondary antibody and washed with 1×TBST for 15 min for a total of three times. Enhanced chemiluminescence (ECL) developers A and B are mixed well at 1:1, the bands are aspirated with filter paper and put into the exposure clip, the freshly prepared developer is added evenly, time of exposure is set and exposure analysis is performed. The results show that with HEK-293T cell lysate as control, the exosomes collected contain the characteristic proteins TSG101 and CD9 and the GAPDH protein content is very low (see FIG. 5D).

### 7. Exosomes Entering Macrophages for Laser Confocal Shooting

### 7.1 PKH26 Fluorescent Dye Labeling Exosomes

(1) the exosomes stored at -80° C. are taken out and thawed on ice;
(2) under light-proof conditions, 1 µL of PKH26 fluorescent dye is taken into a 200 µL PCR tube and diluted 100-fold by adding 99 µL of diluent;
(3) all of the diluted PKH26 dye is added to the exosomes, followed by vigorous vortexing for 1 min to mix and incubation for 20 min protected from light;
(4) 35 mL PBS is added and centrifuged at 4° C. and 100,000 g for 90 min; and
(5) the supernatant is discarded, then the exosomes are res-suspended after adding with 100 µL PBS, then stored at -20° C. in the dark.

### 7.2 Exosomes Treating Kupffer Cells, Slides Preparation and Shooting by Laser Confocal

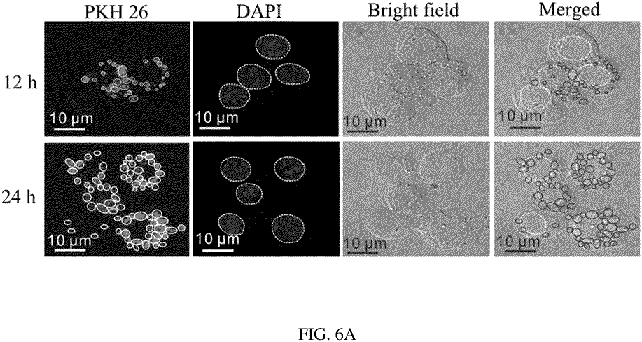

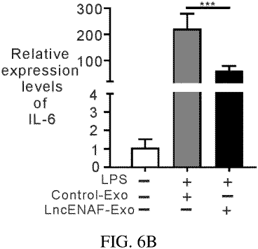

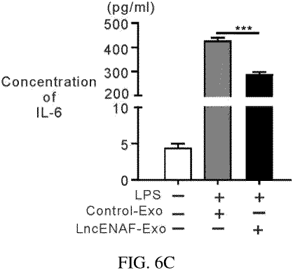

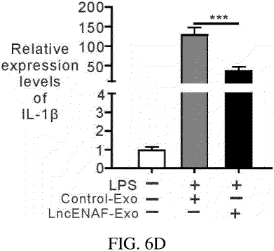

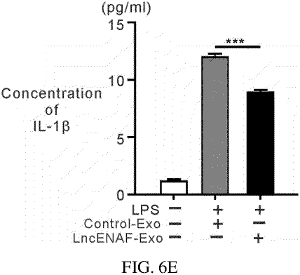

(1) a 24-well plate is taken, and a round cell climbing slice is put into each hole and inoculated with 5×10⁴ cells;
(2) after 12 h of cell culture, 20 µL of fluorescently labeled exosomes are added to each well, and the climbing slice is taken out at 12 h and 24 h of incubation, respectively, followed by adding 500 µL of PBS and shaking lightly several times, discarding the PBS, and repeating three times;
(3) 200 µL of 4% paraformaldehyde is added, followed by fixing at 4° C. overnight;
(4) the fixed climbing slices are taken out, with paraformaldehyde discarded and 500 µL PBS is added, followed by slightly shaking for several times and discarding the PBS; the operation is repeated for three times;
(5) a clean slide is taken and added with a drop of anti-fluorescence quencher, the climbing slice is taken out with forceps, carefully absorbed by touching with filter paper by the edge, and inverted on the anti-fluorescence quencher, with drops of neutral resin around the edge, and left for 30 min at room temperature away from light, then stored at 4° C. after stabilization; the next day, laser confocal observation is conducted and red fluorescence of PKH26 is observed in the blastocytes, indicating that the blastocytes can engulf PKH26-labelled exosomes and that the number of engulfed exosomes increases as the incubation time increases (see FIG. 6A).

### 8. Detection of IL-6 and IL-1β Changes After Co-incubation of Exosomes With Blast Cells and Stimulation With Lipopolysaccharide

(1) HEK-293T-lncENAF cell strain and control cell strain are inoculated into 24-well plates, and each hole is inoculated with 5×10⁴ cells;
(2) the cells are attached to the wall after 12 h, then 25 µg of exosomes are added to each well; after continued incubation for 12 h, lipopolysaccharide at a final concentration of 50 µg/mL is added to each well to stimulate the blighted cells for 24 h; the cell supernatants after 24 h are collected and the concentrations of IL-6 and IL-1β in the supernatants are measured by enzyme linked immunosorbent assay (ELISA) (see 9. ELISA for IL-6 and IL-1β); the changes in mRNA expression of IL-6 and IL-1β in blighted cells are detected using qPCR, and the results suggest that the addition of Exo-ENAF suppresses LPS-induced mRNA expression of IL-6 and IL-1β (see FIG. 6B and FIG. 6D).

### 9. ELISA for IL-6 and IL-1β

Operations: with reference to the IL-6 kit (MuitiSciences: mouse IL-6 ELISA kit (70-EK206/3)) and the IL-1β kit (MuitiSciences: mouse IL-1β ELISA kit (70-EK201B/3)), the details are as follows:
(1) cell culture supernatant: it is centrifuged at 300 g under room temperature for 10 min, and the supernatant is collected after centrifugation;
(2) dilution of the standard: the standard is shortly centrifuged before opening the cover, the mouse IL-6/IL-1β standard is dissolved with distilled water, followed by gently swirling and shaking to ensure full mixing, where the concentration of standard substance is 1,000 pg/mL, and standing for 20 min;
(3) preparation of standard curve for cell culture supernatant samples: 230 µL of concentrated standard is added to a 230 µL cell culture medium as the highest concentration (500 pg/mL) for the standard curve; for each tube, 230 µL of cell culture medium is added; a 1:1 serial dilution is prepared using a highly concentrated standard; it is important to ensure that each pipette is well mixed and that the cell culture medium is used as the zero concentration for the standard curve;
(4) before testing, all reagents and samples are equilibrated to room temperature and all required reagents and working concentration standards are prepared;
(5) unwanted bands are dismantled and returned to the foil bag containing the desiccant and resealed;
(6) immersion of the ELISA plate: 300 µL of 1× washing solution is added and left to soak for 30 s; the washing solution is discarded and the plate is patted dry on absorbent paper (use the plate immediately after the wash is completed and do not allow the plate to dry);
(7) sample addition: the standard wells are supplemented with 100 µL of 2-fold diluted standard, the blank wells are supplemented with 100 µL of medium and the sample wells are supplemented with 100 µL of cell culture supernatant;
(8) addition of detection antibody: 50 µL diluted detection antibody (1:100 dilution) is added to each well; (samples in steps (6), (7) and (8) are added continuously without interruption and the process is completed within 15 min;)
(9) incubation: the plate is sealed with a sealing film, then oscillated at a speed of 300 rpm, and incubated at room temperature for 1.5 h;
(10) washing: the liquid is discarded and 300 µL of washing solution is added to each well to wash the plate for six times, and the plate is patted dry on blotting paper for each wash;
(11) incubation with enzyme: 100 µL of diluted horseradish peroxidase labeled streptavidin (1:100 dilution) is added to each well;
(12) incubation: the plate is sealed with a new sealing film, oscillated at 300 rpm, and incubated at room temperature for 0.5 h;
(13) washing: step (10) is repeated;
(14) development by adding substrate: 100 µL of developing substrate TMB is added to each well, and incubated for 20 min in the dark at room temperature;
(15) addition of stopping liquid: each well is added with 10 µL of stopping liquid, with the color changed from blue to yellow (if the colour appears green or if the colour change is noticeably uneven, tap the frame gently to mix it well);
(16) detection reading: within 30 min, dual wavelength measurement is carried out using an enzyme marker to determine the OD values at the 450 nm maximum absorption wavelength and the 570 nm reference wavelength; the calibrated OD is the 450 nm measurement minus the 570 nm measurement; using only the 450 nm measurement results in a high OD value and reduced accuracy. The results indicate that the addition of exosomes secreted by cells stably expressing lncENAF inhibits the LPS-induced release of IL-6 and IL-10 cytokines (see FIGS. 6C, 6E for results).

The above-mentioned embodiments only describe the preferred mode of the present application, and do not limit the scope of the present application. Under the premise of not departing from the design spirit of the present application, various modifications and improvements made by ordinary technicians in the field to the technical scheme of the present application shall fall within the protection scope determined by the claims of the present application.

What is claimed is:

1. An exosome inhibiting macrophage cytokines, comprising the exosome secreted by a cell strain of human embryonic kidney 293T cells (HEK-293T) obtained by genetic engineering, wherein the cell strain of HEK-293T stably expresses lncRNA elevated in non-alcoholic fatty liver (lncENAF), and the lncENAF has a nucleotide sequence as shown in SEQ ID NO:1.

2. An application of the exosome according to claim 1 in preparing medication for inhibiting increased cytokines levels, comprising inhibiting cytokine storms or treating autoimmune diseases by using the exosome to inhibit increased cytokines levels, wherein the cytokines levels comprise interleukin-6 (IL-6) and interleukin-1 beta (IL-1β).

3. A medication for inhibiting increased cytokines levels or treating autoimmune diseases, comprising the exosome according to claim 1, and a pharmaceutically or immunologically combinable carrier or adjuvant.

4. A method for constructing the cell strain of HEK-293T according to claim 1, comprising:
S1, obtaining a gene sequence of lncENAF and constructing a lentiviral vector for stably expressing the lncENAF;
S2, mixing HEK-293T cells with the lentiviral vector for lentiviral plasmid transfection to obtain virus solution; and
S3, mixing the virus solution with the HEK-293T cells for culture, and obtaining the cell strain of HEK-293T stably expressing lncENAF through antibiotic screening.

5. A usage of the exosome inhibiting macrophage cytokines according to claim 1, comprising steps as follows:
constructing a cell strain of HEK-293T stably expressing lncENAF by the method for constructing the cell strain of HEK-293T according to claim 4, then culturing to collect a culture solution, followed by centrifugation to collect exosomes secreted by the cell strain of HEK-293T, co-incubating the exosomes with macrophages, and detecting expression levels of cytokines of the macrophages.


SEQUENCE LISTING (12 sequences)

SEQ ID NO: 1 - DNA, 653 bp, synthetic construct
  source 1..653: mol_type = other DNA
attgtacaccatgcagacaaagcgctcaaacacttgcaatgctgggctttctcccaatatcctctgccatctttcctacccttataaaagtccagaaagaaaataatcattctatctggaggtgggggccacttctttaatcctggcacttgggaagcagaggtaggtgtattgctttgagttcaagaccagactggtctacaaagtgagttccgggacagtcaggactgttgaacttggaagccttgtcctcaaatttctggcaattttactagcaccagtcttcccgcctcagcctccagtgtcttcctgagatgatctgactgcatgaaatgccctcgcctcattttagttggctggccctaaggtcaaggtaaatccgcgcccaagctgcccggtggaggtggtctcagagggtgctgcgggatcgaggtagtgaggagactagatcgcaagacgtgatcctcacatttatttgcctggagttctcatgccagagaacctggcagattttactatttcccaattgtttactcgccaagctttcaggtccacgcgcctcagggctgcgcctctcactctgaaacttcattcaaaggccaggcagggaggcccaagaggtggcgaatgggcttgagtatgacctcaaggcc

SEQ ID NO: 2 - DNA, 9863 bp, synthetic construct
  source 1..9863: mol_type = other DNA
acgcgtgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacaataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacctgaaagcgaaagggaaaccagagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccactgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcctcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagattggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcggttaacttttaaaagaaaaggggggattggggggtacagtgcaggggaaagaatagtagacataatagcaacagacatacaaactaaagaattacaaaaacaaattacaaaaattcaaaattttatctcgagccatagagcccaccgcatccccagcatgcctgctattgtcttcccaatcctcccccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagtcgctcgagtcaggcaccgggcttgcgggtcatgcaccaggtgcgcggtccttcgggcacctcgacgtcggcggtgacggtgaagccgagccgctcgtagaaggggaggttgcggggcgcggaggtctccaggaaggcgggcaccccggcgcgctcggccgcctccactccggggagcacgacggcgctgcccagacccttgccctggtggtcgggcgagacgccgacggtggccaggaaccacgcgggctccttgggccggtgcggcgccaggaggccttccatctgttgctgcgcggccagccgggaaccgctcaactcggccatgcgcgggccgatctcggcgaacaccgcccccgcttcgacgctctccggcgtggtccagaccgccaccgcggcgccgtcgtccgcgacccacaccttgccgatgtcgagcccgacgcgcgtgaggaagagttcttgcagctcggtgacccgctcgatgtggcggtccggatcgacggtgtggcgcgtggcggggtagtcggcgaacgcggcggcgagggtgcgtacggccctggggacgtcgtcgcgggtggcgaggcgcaccgtgggcttgtactcggtcatagggccgggattctcctccacgtcaccgcatgttagaagacttcctctgccctcgcgagatccggtggagccgggtccggcggtgccgtccacggcagaattggacgactgagcgcgggatctggcgaaggcgatgggggtcttgaaggcgtgctggtactccacgatgcccagctcggtgttgctgtgcagctcctccacgcggcggaaggcgaacatggggcccccgttctgcaggatgctggggtggatggcgctcttgaagtgcatgtggctgtccaccacgaagctgtagtagccgccgtcgcgcaggctgaaggtgcgggcgaagctgcccaccagcacgttatcgcccatggggtgcaggtgctccacggtggcgttgctgcggatgatcttgtcggtgaagatcacgctgtcctcggggaagccggtgcccaccaccttgaagtcgccgatcacgcggccggcctcgtagcggtagctgaagctcacgtgcagcacgccgccgtcctcgtacttctcgatgcgggtgttggtgtagccgccgttgttgatggcgtgcaggaaggggttctcgtagccgctggggtaggtgccgaagtggtagaagccgtagcccatcacgtggctcagcaggtaggggctgaaggtcagggcgcctttggtgctcttcatcttgttggtcatgcggccctgcttgggggtgccctctccgccgcccaccagctcgaactccacgccgttcagggtgccggtgatgcggcactcgatctccatggcgggcaggccgctctcgtcgctctccatggtaagcttgggctgcaggtcgaaaggcccggagatgaggaagaggagaacagcgcggcagacgtgcgcttttgaagcgtgcagaatgccgggcctccggaggaccttcgggcgcccgccccgcccctgagcccgcccctgagcccgcccccggacccaccccttcccagcctctgagcccagaaagcgaaggagcaaagctgctattggccgctgccccaaaggcctacccgcttccattgctcagcggtgctgtccatctgcacgagactagtgagacgtgctacttccatttgtcacgtcctgcacgacgcgagctgcggggcgggggggaacttcctgactaggggaggagtagaaggtggcgcgaaggggccaccaaagaacggagccggttggcgcctaccggtggatgtggaatgtgtgcgaggccagaggccacttgtgtagcgccaagtgcccagcggggctgctaaagcgcatgctccagactgccttgggaaaagcgcctcccctacccggtagaagctagcggctccggtgcccgtcagtgggcagagcgcacatcgcccacagtccccgagaagttgtggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgaccttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgatctagagctagaattgtacaccatgcagacaaagcgctcaaacacttgcaatgctgggctttccccaatatcctctgccatctttcctacccttataaaagtccagaaagaaaataatcattctatctggaggtgggggccacttctttaatcctggcacttgggaagcagaggtaggtgtattgctttgagttcaagaccagactggtctacaaagtgagttccgggacagtcaggactgttgaacttggaagccttgtcctcaaatttctggcaattttactagcaccagtcttcccgcctcagcctccagtgtcttcctgagatgatctgactgcatgaaatgccctcgcctcattttagttggctggccctaaggtcaaggtaaatccgcgcccaagctgcccggtggaggtggtctcagagggtgctgcgggatcgaggtagtgaggagactagatcgcaagacgtgatcctcacatttatttgcctggagttctcatgccagagaacctggcagattttactatttcccaattgtttactcgccaagctttcaggtccacgcgcctcagggctgcgcctctcactctgaaacttcattcaaaggccaggcagggaggcccaagaggtggcgaatgggcttgagtatgacctcaaggccgcgtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggtacctttaagaccaatgacttacaaggcagctgtagatcttagccactttttaaaagaaaaggggggactggaagggctaattcactcccaacgaaaataagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccagttccgcccattctccgccccatggctgactaattttttttatttatgcagaggccgaggccgcctcggcctctgagctattccagaagtagtgaggaggcttttttggaggcctagacttttgcagagacggcccaaattcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagctg

SEQ ID NO: 3 - DNA, 45 bp, synthetic construct
  source 1..45: mol_type = other DNA
tttcaggtgtcgtgatctagaattgtacaccatgcagacaaagcg

SEQ ID NO: 4 - DNA, 43 bp, synthetic construct
  source 1..43: mol_type = other DNA
atccagaggttgattgtcgacggccttgaggtcatactcaagc

SEQ ID NO: 5 - DNA, 25 bp, synthetic construct
  source 1..25: mol_type = other DNA
catccgtaaagacctctatgccaac

SEQ ID NO: 6 - DNA, 19 bp, synthetic construct
  source 1..19: mol_type = other DNA
atggagccaccgatccaca

SEQ ID NO: 7 - DNA, 20 bp, synthetic construct
  source 1..20: mol_type = other DNA
ggaagcagaggtaggtgtat

SEQ ID NO: 8 - DNA, 20 bp, synthetic construct
  source 1..20: mol_type = other DNA
ggcttccaagttcaacagtc

SEQ ID NO: 9 - DNA, 20 bp, synthetic construct
  source 1..20: mol_type = other DNA
cggccttccctacttcacaa

SEQ ID NO: 10 - DNA, 22 bp, synthetic construct
  source 1..22: mol_type = other DNA
ttgccattgcacaactcttttc

SEQ ID NO: 11 - DNA, 22 bp, synthetic construct
  source 1..22: mol_type = other DNA
gaaatgccaccttttgacagtg

SEQ ID NO: 12 - DNA, 21 bp, synthetic construct
  source 1..21: mol_type = other DNA
tggatgctctcatcaggacag